(19) United States
(12) Patent Application Publication
(10) Pub. No.: US 2013/0199540 A1
(43) Pub. Date: Aug. 8, 2013

(54) Device for Plasma Treatment of Living Tissue

(71) Applicant: Christian Buske, Bielefeld (DE)

(72) Inventor: Christian Buske, Bielefeld (DE)

(21) Appl. No.: 13/635,029

(22) PCT Filed: Mar. 16, 2011
(86) PCT No.: PCT/EP11/53928

§ 371 Date: Nov. 15, 2012

(30) Foreign Application Priority Data
Mar. 16, 2010 (DE) 10 2010 011 643.2

(51) Int. Cl. A61B 18/04

(52) U.S. Cl. CPC A61B 18/042
USPC 128/845

(57) ABSTRACT

The invention relates to a device for plasma treatment of living tissue, with a plasma source for generating an atmospheric plasma jet, with a support device for a body part comprising the tissue to be treated, with a movement device for moving the plasma source relative to the surface of the tissue, and with a control device for controlling the movement device and for controlling the operation of the plasma source, wherein the control device has means for adjusting the plasma output as a function of the position relative to the tissue. The invention solves the technical problem of permitting more reliable and faster plasma treatment of living tissue.

The invention concerns a device for plasma treatment of living tissue. In addition a method for operating a device for plasma treatment of living tissue and a method for plasma treatment of living tissue are described. Furthermore, two configurations of plasma sources which are in particular suitable for use in the abovementioned device are described.

In plasma medicine in recent years the collaborative working between plasma physics and life sciences has resulted in the development of some promising applications in the treatment of living tissue. Central to previous plasma applications has been the use of non-thermal atmospheric pressure plasma directly on or in the living tissue. Decontamination which may extend to sterilisation of living tissue, and thus the killing of pathogens on or in a living tissue, is at the forefront here. Plasma treatment of living tissues is not restricted to disinfection, however. Further applications which make use of the properties of the plasma can likewise achieve advantageous effects in medicine. Examples of these will be provided in the description of the invention.

Within the context of the present application, living tissue means any human or animal tissue of a living organism. A tissue which also comprises dead cells or layers of cells is also a living tissue within the meaning of the application. In particular a living tissue is associated with a living organism. Living tissue may also be present in organs removed from an organism, intended for transplantation.

Pathogens are substances or organisms which cause processes in other organisms that are harmful to health. Pathogens can be bacteria, protists, fungi, parasites, viroids, viruses, algae or prions.

The killing of pathogens whilst extensively retaining the living tissue represents a particular challenge for and at the same time a limitation to the prior application in plasma medicine. For in the treatment of living tissue the marginal condition must be met that an increase in temperature of the tissue of only up to approximately 40° C. is tolerated by patients. For above a temperature of 40° C., in particular above 45° C., pain is experienced and serious damage caused to the tissue. Therefore previously the electrical power absorbed by the plasma source has been set in a low range of between 2 and 30 Watts in order to achieve correspondingly low plasma outputs.

In particular, therefore, low-energy atmospheric plasmas have previously been used in plasma medicine. By feeding electrical energy, a weak plasma with a low temperature of less than 40° C. is generated, which in the treatment of the tissue can also be applied for a longer period at one point without leading to excessive thermal stressing of the tissue.

The disadvantage of such a weak plasma is that when it is generated the proportion of UV radiation is relatively high. The amount of irradiated UV energy must be minimised, however, because of the long-term impairment of the tissue. This reduces the length of time for which the low-energy plasma can be applied. The result is that treatments with plasma must be spread over many sessions and in each case a not inconsiderable amount of time is required. The treatment lengths are therefore too great on two counts.

A further problem is the high concentration of ozone during the treatment with the low-energy plasmas, since on the one hand with this type of plasma generation a higher proportion of ozone from the plasma source will be given off and on the other due to the manual application a suitable drawing off of the aggressive ozone gas is not possible in an adequate manner. Therefore a special working gas such as argon is used in place of air, since it is easily ionisable and has an advantageous effect on the radiation temperature of the plasma jet.

Furthermore, the previous plasma applications in medicine have been based on manual execution of the individual treatments. For this the plasma source, often in the form of a pen, is brought into proximity with the tissue to be treated manually by a person and the treatment is carried out freehand. Unevenness in the distances between source and tissue and uneven applications over the surface are the result. The quality of the treatment often suffers as a result. Furthermore the plasma sources are required to ensure that the plasma across the full area in front of the source does not exceed a temperature of in particular 40° C. in order that in the event of unintentional approaching of or even contact with the tissue injuries are avoided.

In the state of the art there are also atmospheric plasma systems, with which a relatively cool plasma with a high chemical reactivity can be generated. Since, however, this plasma according to the operating conditions, because of plasma temperatures of above 40° C. and more, often also higher than 100° C., cannot be applied in a stationary manner to the tissue because this would result in burns, there has to date been no possibility for using these plasma sources in the area of medicine. Such plasma sources are known from documents EP 0 761 415 A2, EP 0 986 939 A1, EP 1 067 829 A2, EP 1 230 414 A1, EP 1 236 380 A1, EP 1 335 641 A1 and WO 2008/074604.

The technical problem for the invention is therefore to provide a device and a method which allow a more reliable and more rapid plasma treatment of living tissue. A further technical problem is the provision of a plasma source with improved distance control. Similarly, a technical problem for the invention is to prevent an excessively strong thermal stressing of the tissue during a plasma treatment.

The problems set out above are solved in accordance with the invention by a device with the features of Claim 1.

An initial teaching of the present invention in accordance with Claim 1 concerns a device for plasma treatment of living tissue
with a plasma source for generating an atmospheric plasma jet,
with a support device for a body part comprising the tissue to be treated,
with a movement device for moving the plasma source relative to the surface of the tissue, and
with a control device for detecting the position of the tissue to be treated and for controlling the plasma source for performing the plasma treatment of the tissue,
wherein the control device has means for adjusting the plasma output as a function of the position relative to the tissue.

A second teaching of the present invention concerns a method for operating a device for plasma treatment of living tissue
in which an atmospheric plasma jet is generated with a plasma source,
in which the plasma source is moved with a movement device relative to the surface of the tissue,
in which the movement device is controlled by a control device and
in which the operation of the plasma source is controlled by the control device.

A third teaching of the present invention concerns a method for plasma treatment of living tissue
in which an atmospheric plasma jet is generated with a plasma source,
in which the plasma source is moved with a movement device relative to the surface of the tissue,
in which the movement device is controlled by a control device,
in which the operation of the plasma source is controlled by the control device and
in which the plasma jet acts upon the tissue and at least in part causes the death of pathogens on or in the tissue.

The teachings described above, which are closely tied to one another, are explained in further detail below in a combined description of the individual characteristics and individual features and advantages of the process steps.

The term plasma source means a source for a plasma jet directed at a region, wherein the form of the plasma jet can be designed to be round or flat. The plasma source can also generate a rotating plasma jet, in that a housing part, in particular the outlet opening is designed to move rotationally and rotates during the plasma generation about an axis. The plasma source can have one plasma nozzle or a plurality of plasma nozzles arranged alongside each other in the form of a plasma shower. The plasma source has a support for positioning on the movement device and feeders for working gas and electrical voltage.

The excitation of the working gas can take place at various frequencies, for example in the microwave range in the range of or above 1 MHz or in the frequency range 1-100 kHz. The voltage forms can vary between alternate voltages and pulsed direct voltages. The discharges are microwave discharges or high-frequency radio discharges, which can take the form of discharge arcs (arc discharges) or brush discharges. The voltage amplitudes and frequencies are matched to the plasma nozzle geometry in each case and are by way of example in the range 100 V-10 kV. The working gas used is preferably air, but additionally nitrogen and noble gases such as argon, including in mixtures with other gases such as hydrogen, are possible.

Such plasma sources can generate relatively cold plasma jets with relatively high chemical excitation energy. The radiation temperature drops as a function of the distance from the outlet opening and is in the range from below 300° C., in particular below 150° C. and preferably below 100° C. Depending on the distance from the outlet opening, therefore, temperatures of the plasma jet upon contact with a tissue (or another object) can be maintained in the range below 80° C., 60° C. and 40° C., without the chemical reactivity having dropped too sharply. The high excitation energy of the working gas is the result of the high frequency excitation in the excitation zone within the plasma source, where only a low thermal excitation takes place. A non-thermal plasma is also referred to, therefore. By selecting a suitable working gas the temperature of the plasma jet can also be influenced.

The sources described above also generate a considerably smaller proportion of UV light in relation to the plasma output of the plasma jet than is the case with the plasma sources used to date in the field of medicine. Furthermore, during the plasma generation less ozone is produced since the ozone gas resulting in the plasma from the discharge immediately reacts and is converted due to the higher plasma output.

With plasma jet temperatures above that from which a person will experience pain, thus for example above approximately 40° C.-50° C., it is necessary to move the plasma source in relation to the tissue, in particular to move this in a defined and automatic manner, so that it can be avoided that the plasma source remains stationary relative to the tissue for a long period and the plasma treatment leads to an excessive tissue temperature. Therefore the device has a support device for a body comprising the tissue to be treated, a movement device to move the plasma source relative to the surface of the tissue and a control device to control the movement device and to control the operation of the plasma source.

In this way it is possible, despite the plasma temperatures being above the specified temperature limit before pain is experienced of approximately 40° C.-45° C., to use the plasma sources described above. The more intensive plasma with the features of a smaller proportion of UV light and less ozone generation for the plasma treatment can lead to lower treatment times with greater effectiveness and lower damage to the tissue. Larger areas can similarly be treated and thus the use of plasma treatment can be extended to applications that were not previously possible in plasma medicine.

Compared with the plasma sources known from the state of the art, the higher plasma output means that the energy contained in the plasma jet is sufficient in order to allow chemical reactions or a release of the molecular bonds in the pathogens to an extent that has not previously been possible. Thus a level of disinfection and sterilisation is achieved which could not be arrived at with the previous low-energy plasma sources, since only with the energies of the plasma sources used here can the corresponding chemical reactions be triggered.

The present device is not limited to the application of plasma sources described above. Other plasma sources, for example with other excitation mechanisms, can also be applied, even if their plasma jets have lower temperatures and lower plasma outputs, which make a regular movement of the plasma sources relative to the tissue unnecessary. Even with such known plasma sources the device according to the invention can be advantageously employed. In these cases also a more even and/or faster plasma treatment of the tissue can be achieved.

The support device for the body part comprising the tissue to be treated serves to accurately and reliably position the body part. Because when performing the plasma treatment, because of the features of the plasma jet described above, it is a matter of controlling the distance between the plasma source and the tissue within narrow ranges. Manual guidance of the plasma source is therefore generally unsuitable.

The movement device serves for defined movement of the plasma source relative to the surface of the tissue, so that an automatic and thus reproducible movement pattern of the plasma source relative to the tissue can be guaranteed. To this end the movement device has adjustment and drive mechanisms which allow at least a two-dimensional, in particular three-dimensional, movement of the plasma source relative to the body part or the tissue. Here the number of degrees of freedom to within which the plasma source can be adjusted can vary between two and six. The more degrees of freedom that are available, the more accurately the surface of the tissue to be treated with the plasma source can be scanned. Here it is possible that on the one hand only the plasma source is moved, while the body part remains stationary. On the other hand, the body part can also perform at least part of the movement relative to the plasma source.

Furthermore, a control device for controlling the movement device and for controlling the operation of the plasma source is provided for. As a function of a predefined movement and treatment pattern the relative movement between the plasma source and the body part or tissue is performed, wherein in particular a suitable distance control is applied, which feeds into the control of the movement pattern. Additionally, the control device also controls the method of operation of the plasma source in that for example the plasma source can be switched on and off.

Furthermore, means for adjusting the plasma output as a function of the relative position of the tissue are provided for, so that the plasma output can be adjusted as by influencing the electrical parameters of voltage and current flow and the frequency of the voltage and of the gas flow through the plasma source as a function of the respective position relative to the tissue.

By means of the control device therefore at least a partially automated process during the plasma treatment is made possible, which can ensure that the treatment is reproducible, accurate and meets the desired purpose. Here the plasma jet impinges on the tissue in such a way that to some extent at least killing of pathogens on or in the tissue is brought about, without excessive heating of the tissue resulting. This because the constant movement of the plasma source relative to the tissue prevents the plasma jet impinging for too long on one point of the tissue and as a result of its radiation temperature above the limiting temperature transmitting excessive heat energy onto and into the tissue.

The device according to the invention described above for the plasma treatment of living tissue can be used for disinfection of body parts such as arms, legs or the head, or also parts of the trunk such as the stomach or back. The support device must then be adapted to the shape of the respective body part. The invention also covers the treatment of the entire body of a person, however, if for example a large-area skin complaint has to be treated or if cleaning of a body by disinfection is necessary. Here the support device can also take the form of a standing surface upon which the person to be treated stands.

The device explained above can also be used independently of the plasma treatment of tissue for the cleaning of persons in contaminated protective suits. Therefore in connection with the present invention protection is also sought for a device for a plasma treatment of a protective suit worn by a person,
with a plasma source for generating an atmospheric plasma jet,
with a support device for the person,
with a movement device for moving the plasma source relative to the surface of the protective suit, and
with a control device for detecting the position of the protective suit to be treated and for controlling the plasma source for performing the plasma treatment of at least part of the protective suit.

The invention can thus be used wherever a sealed-off clean area is required, wherein the person wearing the protective suit prior to entering the area undergoes plasma treatment in order to clean the surface of the protective suit. Similarly, the invention can be used wherever in a sealed-off area hazardous substances, for example radioactive materials, chemicals or biological materials, are used. Before leaving the sealed-off area the plasma treatment of the protective suit of the person, or at least a part of this, can take place in order to lessen or prevent the danger to the environment.

All the following configurations and embodiments of the device for plasma treatment of tissue can likewise be used in the context of the application for cleaning of protective suits, wherein in the following description in each case the term “tissue” or “body” or “body part” should be replaced by “protective suit” or “part of a protective suit” or “surface of a protective suit”, respectively.

In the following preferred configurations of the individual device and method features are explained.

In a preferred manner the support device has a positioning device for fixing of the body part. By fixing the body part resting on the support device random movements are prevented, and thus it is ensured to the greatest possible extent that any random movement of the body part leads to a smaller distance between the plasma source and the tissue. To this end a template is preferably used which is detachably secured to the support device.

In a further preferred manner at least one switch is provided for, which opens after a minimum movement of the body part and thus a part of the positioning device or the template. This switch signal can be evaluated by the control device in order as necessary to immediately switch off the plasma source and move it away.

In addition, it is further possible for the positioning device to have at least one movement sensor which detects a movement of the body part to be treated and transmits a corresponding signal to the control device. Here the movement sensor can be selected in a suitable manner; so for example capacitive, inductive or optical movement sensors may be used.

In a further preferred configuration of the invention the movement device moves the plasma source three-dimensionally. In this way scanning of an area of a body part to be treated is also possible in one process cycle even if the surface of the tissue is uneven, without the position of the plasma source having to be readjusted between two parts of the treatment at different times.

Here it is further preferred that the movement device sets the direction of the plasma jet relative to the surface of the tissue. Together with a three-dimensional movement of the plasma source this results in four degrees of freedom.

It is similarly possible for the movement device to move the plasma source in a circular fashion. In this way larger areas undergo the plasma treatment, and the movement device only has to travel smaller distances. In addition, the outlet opening of the plasma source itself can perform a rotating movement. In this way less mass is shifted and the rotating movement can be performed more quickly and more efficiently.

In a further configuration a distance control device checks the distance between the plasma source and the tissue. To do this the distance control by the device can take the form of optical distance measurement, in particular laser distance measurement with electronic distance measurements using the propagation time or phase shift measurement of light, usually laser light.

Furthermore the distance control device can also be designed to provide imaging distance measurement. In this case a camera monitors the area between the plasma source and the tissue to be treated and by means of continuous image interpretation the respective distance can be measured.

The distance control device explained above transmits the distance signal generated in each case to the control device, which as a function of this distance signal adjusts the distance between the plasma source and the tissue. For this the control device can operate the movement device in such a way that the entire plasma source is moved.

The distance adjustment can also be carried out by a plasma source with a variably adjustable length for setting the distance between the plasma source and the tissue. For this the plasma source is designed in a particular manner, and this is described in more detail below.

A further embodiment of the device described to this point has a temperature control device which checks the temperature of the tissue to be treated. The temperature measurement preferably takes place contact-free by measurement of the thermal radiation. This can mean, inter alia, a fibre-optic temperature measurement, in which optoelectronic devices are used for measurement of the temperature, wherein glass fibres are used as sensors for collection and transmission of the thermal radiation.

So, depending on the specific configuration of the device the control device can control the operating parameters of the plasma source and the movement device as a function of at least one of the preset parameters of plasma output, distance, temperature, tissue type and desired effect. An automated process of a plasma treatment with reproducible accuracy and high plasma outputs and plasma energies with short treatment times can therefore be achieved.

A further configuration of the device described is characterised in that a housing is provided in which the plasma source and the movement device are arranged. By screening the housing it is possible to increase patient acceptance of the device, because they have less perception of the technology and similarly, as for a recognised technology, such as with CT scans, a screened environment can be maintained when treatment is being performed. Therefore the housing is preferably designed as a tunnel-shaped treatment area.

Within such a housing the movement device can have a curved guide for circumferential movement of the support for the plasma source, which allows an arched, in particular part circular, movement of the plasma source in a single plane. The support for the plasma source can be moved along the curved guide around a body or a body part, wherein the movement device also has means for changing the radial position, in order to be able to adjust the distance between the plasma source and the tissue to be treated.

Furthermore the movement device within the housing can have a linear guide on which the curved guide is arranged, in order to be able to perform a translation movement transversally to the plane of the curved guide. Thus in addition to the movement of the plasma source in the plane of the curve a relative movement along the body can be carried out.

In particular the housing can also have a suction device in order to draw off the gases resulting during the plasma treatment. In this way, inter alia, even if in lower quantities, ozone generated in the plasma source is drawn off without any interference with the patient or the environment resulting.

As an alternative to the curved guide mentioned above, it is also possible for the movement device to have a robot arm to which the plasma source is secured. This robot arm can be arranged both in a housing in place of the curved guide and possibly the linear guide within the housing described. The robot arm can also be used without a housing, for example for treatment at difficult to access points of the body which cannot be reached within a movement device within a housing. Similarly the use of a robot arm is possible before, during or after operations where use of a housing would be impossible or difficult.

The device described above for performing plasma treatment of living tissue can be operated in the following manner.

Initially it is preferred that the operating parameters of the plasma source and the movement device are controlled as a function of at least one of the preset parameters of plasma output, distance, temperature, tissue type and desired effect.

For example means, in particular on the control device, can be provided so that a certain plasma output can be set, which is to be applied to the tissue to be treated. Then during treatment the plasma source is operated, i.e. the electrical parameters and/or the gas flow parameters are adjusted, such that as a function of the distance from the tissue and/or the relative speed between the plasma source and the tissue this plasma output is transmitted.

Similarly or alternatively temperature monitoring can be provided which when a specified tissue temperature is exceeded triggers a reduction in plasma output or the switching off of the plasma source. Alternatively the plasma source could also be moved away from the tissue as quickly as possible, without the operating parameters of the plasma source being varied. A combination of a variation of the operating parameters and moving away can also be applied.

If for the different treatment of various types of tissue different plasma outputs and intensities of the plasma treatment are necessary, then in particular on the control device a corresponding selection possibility for different plasma treatments is provided for. The control device will then operate the plasma source and the movement device as a function of the setting. It can similarly be provided that individual parameters for influencing the plasma treatment can be adjusted separately. This makes individualised plasma treatment possible.

For the control of the movement of the plasma source there is initially the possibility that the absolute position of the section of the tissue to be treated is determined and that the movement and operation of the plasma source are then carried out automatically. This method requires determination of the position only to begin with, after which the plasma treatment is carried out using the data determined at the outset. Here therefore continuous distance measurement is unnecessary, but it must be ensured that the area of the tissue to be treated does not move. Determination of the position can for example take place optically using a camera or by scanning with a distance measurement device. The measured data are stored in the control device and then used as a basis for control of the plasma source during the plasma treatment. In order to perform this process step the device for plasma treatment of living tissue has suitable means for determining the absolute position of the tissue.

Alternatively, it is also possible that during the movement and operation of the plasma source the position of the tissue relative to the plasma source is determined and the plasma source is controlled by the control device as a function of this relative position. This alternative therefore requires an active distance measurement, the measured data of which are fed directly into the control of the movement device and the plasma source. In order to perform this process step the device for plasma treatment of living tissue has suitable means for determining the position of the tissue relative to the plasma source during the movement and operation of the plasma source.

Furthermore and in particular the method can be carried out in such a way that a possible movement of the body part is monitored and the amplitude of the movement determined, and that the plasma treatment is interrupted if an amplitude of the movement above a limiting value is detected. For if this amounts to an intended or unintended movement of the body part then it must be ensured that an excessive plasma effect on the tissue cannot occur. Interruption of the plasma treatment means that the plasma output is reduced or switched off and/or the plasma source is moved away from the body.

In a further configuration the control device now has means for establishing an output profile within the area to be treated, so that prior to the plasma treatment the plasma output profile can be established within the area to be treated. With this measure the plasma treatment can be set in a targeted manner as a function of the state of the tissue and the treatment intensity can be changed in a variable manner across the surface. For especially with large area plasma treatment of diseased or damaged skin areas the output profile can be selected according to the required intensity of the plasma treatment and thus an individualised treatment applied.

The method for plasma treatment of living tissue corresponds essentially to the method described above inclusive of the preferred configurations thereof. The method is in particular characterised in that the plasma jet acts upon the tissue and at least in part causes the death of pathogens on or in the tissue. The advantage of the plasma treatment is that despite the effect of the energy no lasting damage to the tissue is caused by the plasma, but the killing effect on pathogens is still ensured. The energy of the plasma jet is sufficient to kill pathogens, wherein this energy also leads to damage in the tissue layers. But since the body's self-healing capabilities are sufficient to reproduce the damaged tissue layers, the healing process is improved since the aggressive pathogens have been reduced or even eliminated.

Through the use of the plasma sources described above when the method is performed an automated process is employed which is performed without direct intervention by a treating person. Despite plasma jet temperatures that are above the limiting value of approximately 40° C.-45° C. therefore an intensive and accurate plasma treatment can be applied.

Prior to performing the plasma treatment it is preferred to determine the precise dimensions of the area of the tissue to be treated. In this way it can be ensured that the plasma treatment is not extended to areas that are not to be treated. Similarly, the topography, that is to say the three-dimensional surface form, can be determined. The determination of the area to be treated and possibly its topography can be performed with optical means, for example a camera, wherein on a display device, within the camera image captured selection of the area can be performed by the treating person.

Furthermore, prior to performing the plasma treatment, the distance between the plasma source and the tissue can be specified and entered as a parameter. Then, during the plasma treatment, the distance between the plasma source and the tissue can be adjusted, preferably continuously, within a specified range, possibly also on the basis of the measured topography of the area.

An advantageous measure is also if the temperature of the tissue to be treated is determined before and/or during and/or after the plasma treatment. In this way the critical parameter of a possible overheating of the tissue is monitored and burns from the plasma treatment are avoided. Here the plasma treatment can be interrupted by the control device if the absolute temperature or a temperature difference exceeds a specified limiting value during treatment.

Similarly, a possible movement of the area of the tissue can be monitored and the amplitude of the movement determined, with the plasma treatment being interrupted if an amplitude of the movement above a limiting value is detected.

Above, the plasma treatment has been described as a direct application of a plasma jet to the tissue. In the following, further measures are described which can be carried out additionally or alternatively to the direct plasma treatment.

Before, during and after the plasma treatment a heat treatment, light treatment and/or a laser treatment can be carried out. These additional measures can support and extend the way in which the plasma treatment works.

In a preferred manner after plasma treatment the tissue can be sealed. In this way recontamination with pathogens can be avoided or sharply reduced. The seal here can be any synthetic or natural layer. Here tissue or non-tissue such as smooth layers can be applied.

In a particularly preferred manner the material of the seal is generated by the plasma source through plasma polymerisation. During the plasma polymerisation, with the help of a plasma, a precursor material is reacted and the reacted product deposited onto the surface. Here both the reaction and the depositing take place under atmospheric pressure. Since the precursor material is in particular fed and introduced into the plasma jet separately from the working gas, the precursor material itself does not need to pass through the entire excitation zone. This has the important advantage that the precursor material does not decompose or is not otherwise chemically altered as early as the excitation zone. For the desired reaction, which leads to the depositing of a polymer-like layer on the surface of the substrate, therefore a considerably larger number of reaction partners are available than with the normal process. Such a process and a corresponding device are known from EP 1230414 A1.

The depositing of a seal onto a body tissue by means of plasma polymerisation constitutes a process that is independent of the previously performed plasma treatment of the tissue for which protection will be sought in its own right.

The tissue seal can thus be applied either directly after or actually during the plasma treatment, so that any time delay between plasma treatment and application of a seal can be excluded or at least considerably reduced. Where the plasma treatment and plasma polymerisation take place simultaneously, it can be difficult to distinguish between the two processes since the plasma jet is not applied during plasma polymerisation. The plasma treatment and the plasma polymerisation can thus be performed with the same plasma source. Following an interruption to the feed of the precursor material a plasma jet without reaction products can be generated, and vice versa.

One advantage of the application of a seal to the tissue by means of plasma polymerisation is that the layer deposited is very thin and has only a minor adverse effect on the body. Seals that are breathable but at the same time repel pathogens can thus be produced.

Furthermore, in a preferred manner following the plasma treatment or independently of a prior plasma treatment a medication can be applied to the tissue and the medication activated by the plasma jet. Here activation means any form of influencing the effectiveness of the medication through heat and/or chemical excitation and/or depositing of an additional component of the medicament by plasma polymerisation.

Furthermore, by means of plasma polymerisation the medication itself can also be deposited. In this case prior to the plasma treatment there is no medication on the tissue, but during the plasma treatment the medication is applied. This results in an effective application of medications and possibly an enhancement of their action. Because the medication can not only be deposited on its own but can also be activated by the interaction with the plasma.

The plasma treatment described above of living tissue can be used for disinfection of tissue that has been contaminated by an injury or infected by pathogens. Where the injury is fresh, the plasma treatment can be used prophylactically and if there is already contamination and the tissue is inflamed then the plasma treatment can help to cure the inflammation.

In a particularly preferred manner prior to the start of an operative intervention the operating site on the body can be disinfected with the plasma treatment. In this way the disinfection with chemical agents used to date can be supplemented, supported or even replaced. It is similarly possible that before and after closing an operating site the wound area closed or to be closed is disinfected with plasma treatment. In this way the risk of inflammation by pathogens can be reduced.

The device described above for plasma treatment of living tissue and the associated method are based on the use of plasma sources which generate a low-temperature, non-thermal plasma jet. The two configurations of the plasma source that are described below in terms of the variation in length of the plasma source and the distance measurement integrated into the plasma source can in each case be used by preference in this device and this method. The configuration of the plasma sources is not restricted to this use, however. Therefore, both configurations constitute independent inventions which can be used quite generally in plasma treatments and plasma applications.

In a further independent teaching of the present invention the plasma source for generating an atmospheric plasma jet has a design with a support, with a housing, with an internal electrode, with an external electrode that is at least partially formed in the housing, with a gas inlet, with an outlet opening formed on the housing and with means for applying a high-frequency high voltage between the internal electrode and the external electrode, wherein the internal electrode and the outlet opening together define an axial direction. This configuration is characterised in that the position of the external end of the outlet opening can be varied in the axial direction relative to the support.

Through this variability of the length of the plasma source a rapidly adjustable control of the distance between the outlet opening of the plasma source and the surface of the object or tissue to be treated is possible. For the mass to be moved restricts the possible frequency of the variation in length. Since only part of the plasma source and no longer the plasma source together with the support has to have its axial position changed, because of the low mass to be moved a higher adjusting speed can be achieved.

The rapid control of the length of the plasma source can be used in an advantageous manner in the plasma treatment of uneven surfaces if the unevenness has a typical variation in length that is greater than or equal to the dimension of the plasma jet and if during passing over the surface tracking of the distance is required. In this way an even application to the surface of the plasma jet is achieved since the characteristics of the plasma jet can vary with the distance.

A number of possibilities arise for the variation in length of the plasma source.

In a preferred manner the housing of the plasma source has a variable length in the area of the outlet opening. For this purpose the housing is provided with a separate mouthpiece at the front end, which by means of a motor or pneumatically can be displaced relative to the remainder of the housing. For this on the one hand a rotary drive can be used which displaces the mouthpiece by the turning of a thread. On the other, a linear drive can also be used which displaces the mouthpiece by means of a telescopic arrangement which is formed between the housing and the mouthpiece. The advantage of this configuration is that only a small weight is displaced, so that the movement can take place quickly.

In a further alternative configuration of the plasma source the housing is length-adjustable in the axial area between the internal electrode and the outlet opening. Here therefore the mouthpiece is not provided so that it moves, but a section of the housing upstream of the mouthpiece or the outlet opening is designed in two parts, wherein the two separate housing sections relative to one another are moved by a linear drive or a rotary drive towards each other. This configuration has the advantage that the area of the mouthpiece that is important for the discharge process does not have to have its geometry changed rather it is the housing outside of this sensitive area that is altered. Even if more mass is moved, the frequency of the displacement movement is always sufficient for most applications and greater than if the entire plasma source has to be moved.

In a further preferred configuration of the plasma source, the position of the housing together with at least part of the internal electrode relative to the support is variable. For this, by way of example, both the housing and also the internal electrode have a two-piece design and can be displaced in pairs in relation to one another. The front part of the housing is then displaced via a mechanical connection together with the front part of the electrode relative to the two other parts of the housing and the internal electrode. Here both a rotary drive and also a linear drive can be used. With this configuration it is particularly advantageous that the overall geometry of the area responsible for the discharge within the plasma nozzle is not changed during the movement. For the distance between the front end of the internal electrode and the outlet opening remains the same during the movement. Even if in this configuration more mass is moved than in the embodiments explained previously, the frequency of the displacement movement is still sufficient for most applications.

In a further independent teaching of the present invention the plasma source for generating an atmospheric plasma jet has a design with a housing, with an internal electrode, with an external electrode that is at least partially formed in the housing, with a gas inlet, with an outlet opening formed on the housing and with means for applying a high-frequency high voltage between the internal electrode and the external electrode, wherein the internal electrode and the outlet opening together define an axial direction. This configuration is characterised in that means for coupling a laser beam in the axial direction are provided and in that optical means measure the distance between the front end of the outlet opening and the object to be treated, wherein a signal from the reflection of the laser beam from the surface of the object is evaluated.

In a preferred manner the means for coupling the laser beam take the form of a channel created in the internal electrode. The laser beam then runs through the internal electrode, through the housing and through the outlet opening as far as the surface of the object to be treated. The reflected light follows the same path back through the plasma source and is then decoupled from the received signal. The distance information is then gleaned from the pulsed signal and its propagation time and phase displacement.

In a further preferred embodiment the means for coupling the laser beam are in the form of fibre optics running through the internal electrode. The channel described above in the internal electrode is not used for transmitting the free laser beam but for accommodating the fibre optics. In particular the fibre optics run as far as the front end of the internal electrode, but the end of the fibre optics can also end before the front end of the internal electrode. Through the use of fibre optics the coupling, in particular for a rapidly moving plasma source, is simplified compared with coupling using an arrangement of mirrors.

For the evaluation of the reflected light signal the means for distance measurement have a light-sensitive detector and an evaluation device. These work in the normal manner.

In the following the invention is explained using embodiments. The drawing shows as follows:

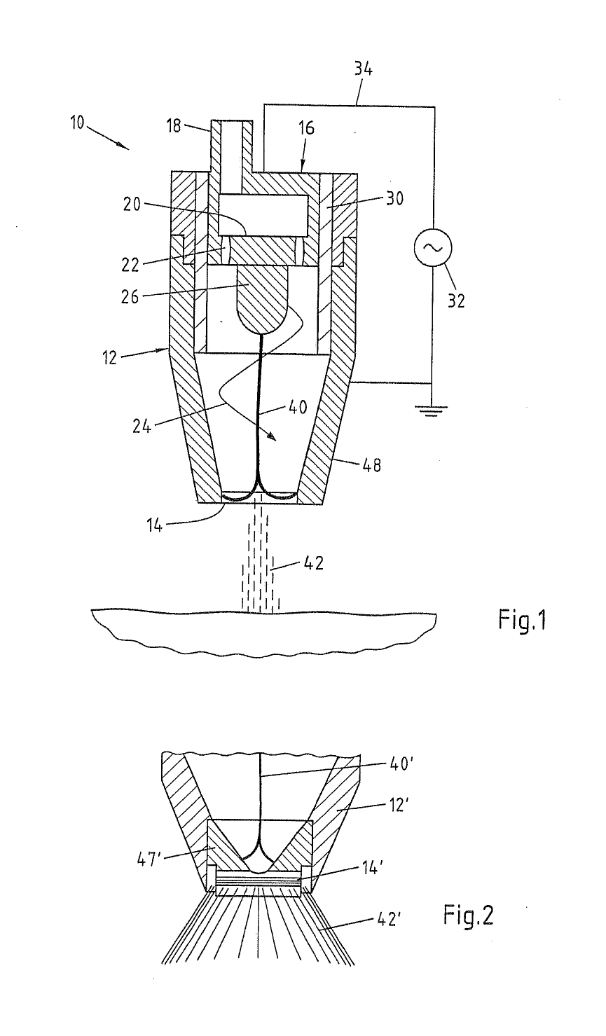

FIG. 1 an embodiment of a plasma source for generating a plasma jet (state of the art);

FIG. 2 in detail, a further embodiment of a plasma source for generating a plasma jet with a slotted outlet opening (state of the art);

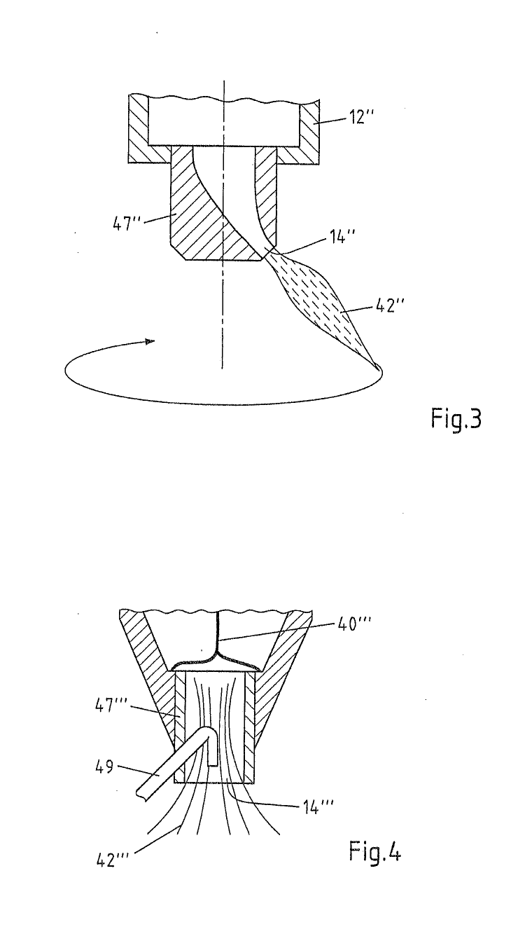

FIG. 3 in detail, a further embodiment of a plasma source for generating a rotating plasma jet (state of the art);

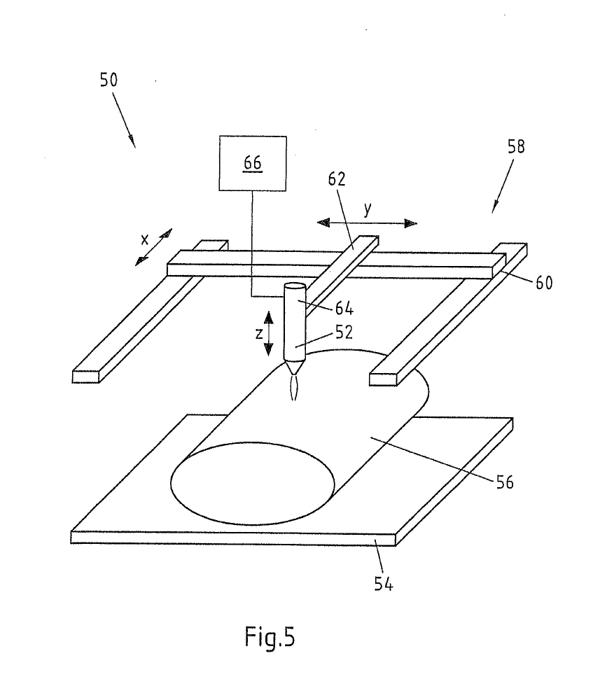

FIG. 4 in detail, an embodiment of a plasma source for generating a plasma jet for plasma polymerisation (state of the art);

FIG. 5 a first embodiment of a device according to the invention for plasma treatment of living tissue with a movement device for 3-dimensional movement of the plasma source with linear displacement directions;

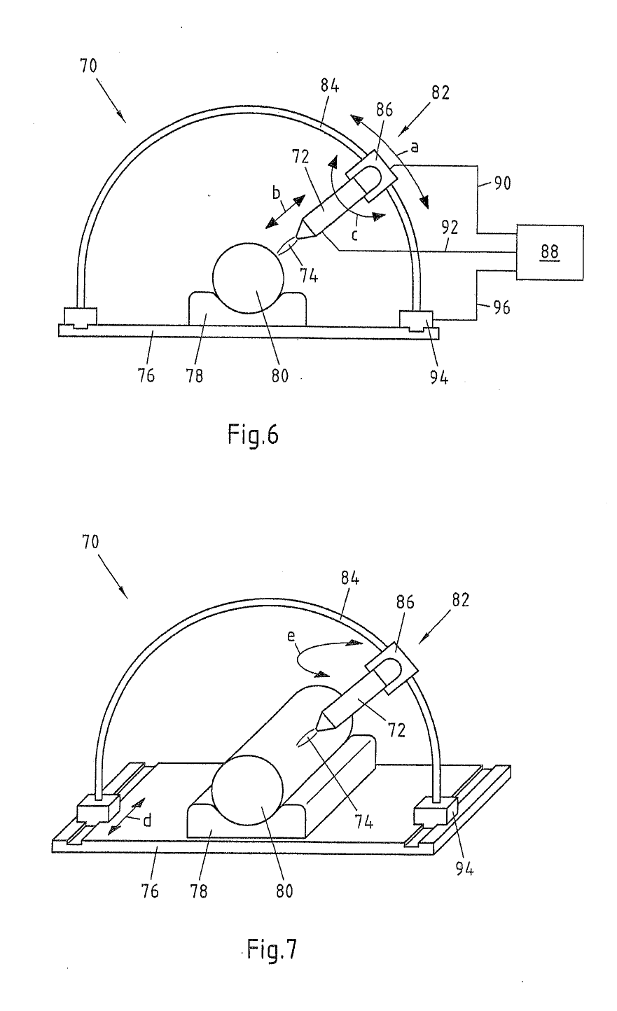

FIG. 6 a second embodiment of a device according to the invention for plasma treatment of living tissue with a movement device for 3-dimensional movement of the plasma source with a combination of curved and linear displacement directions;

FIG. 7 a third embodiment of a device according to the invention for plasma treatment of living tissue with a movement device according to FIG. 6, wherein the alignment of the plasma source can be tilted;

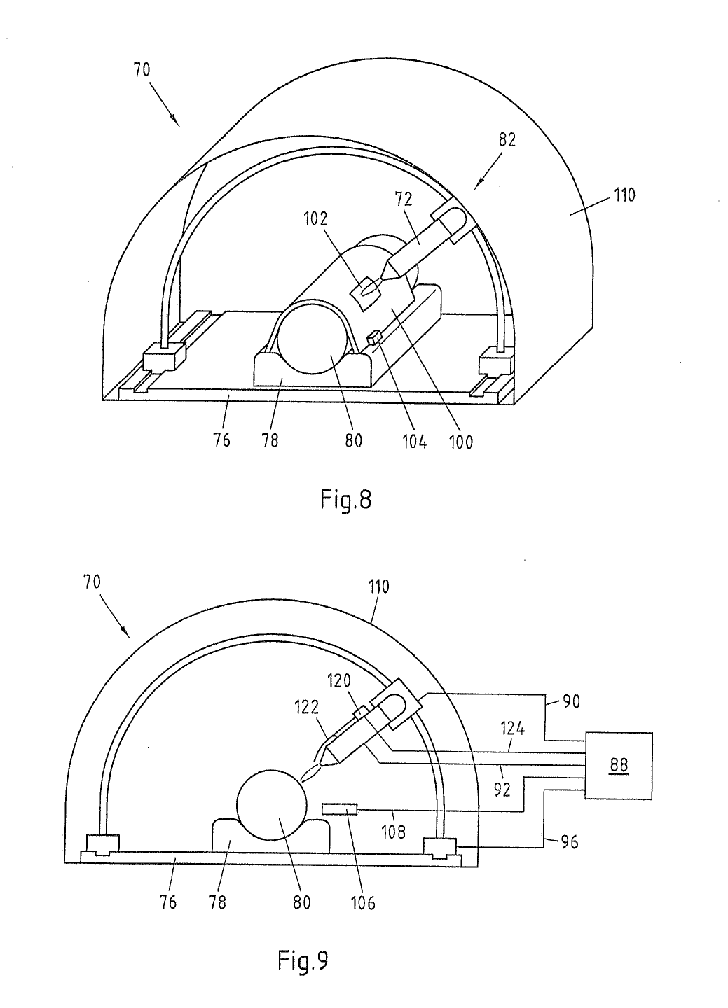

FIG. 8 a fourth embodiment of a device according to the invention for plasma treatment of living tissue according to FIG. 6 or FIG. 7 with a housing and a template as a fixing device;

FIG. 9 a fifth embodiment of a device according to the invention for plasma treatment of living tissue according to FIG. 8 with a temperature control device;

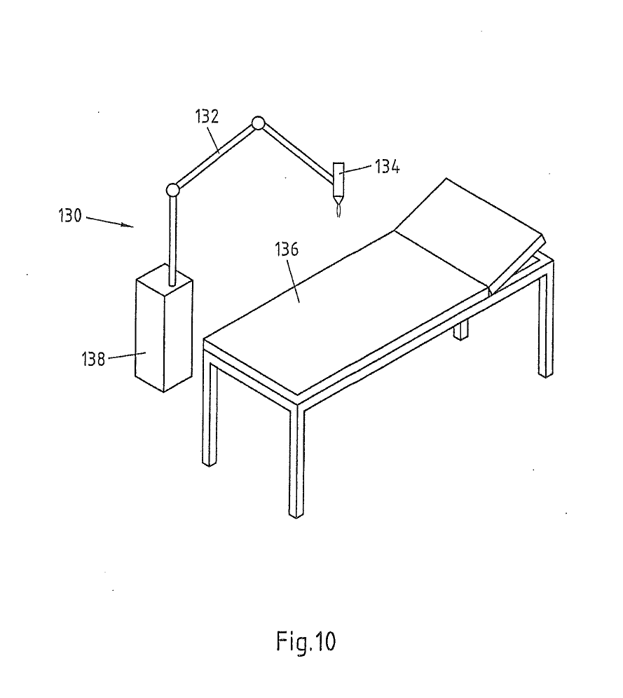

FIG. 10 a sixth embodiment of a device according to the invention for plasma treatment of living tissue with a movement device in the form of a robot arm;

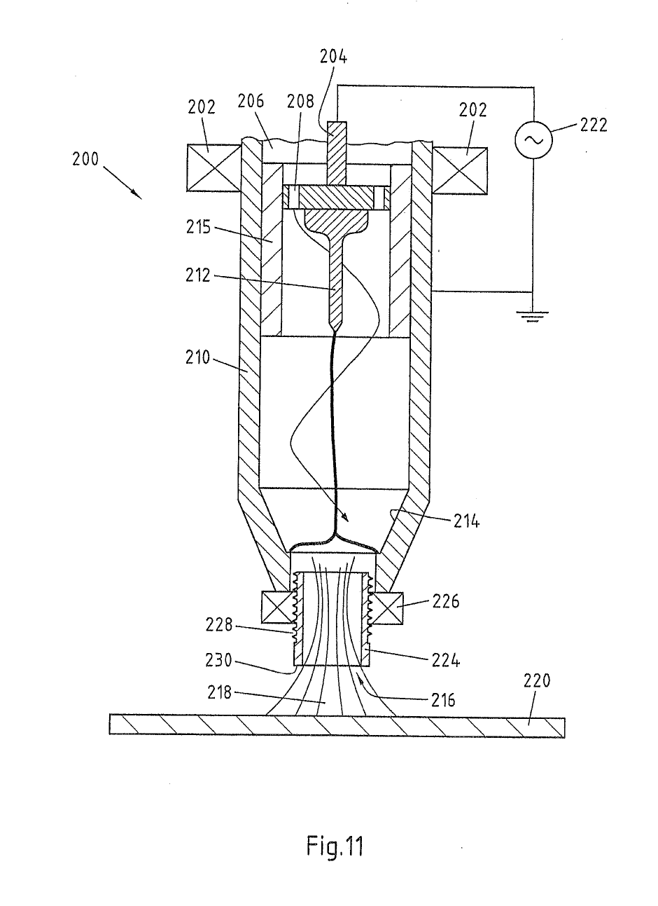

FIG. 11 a first embodiment of a plasma source with an axially displaceable outlet opening;

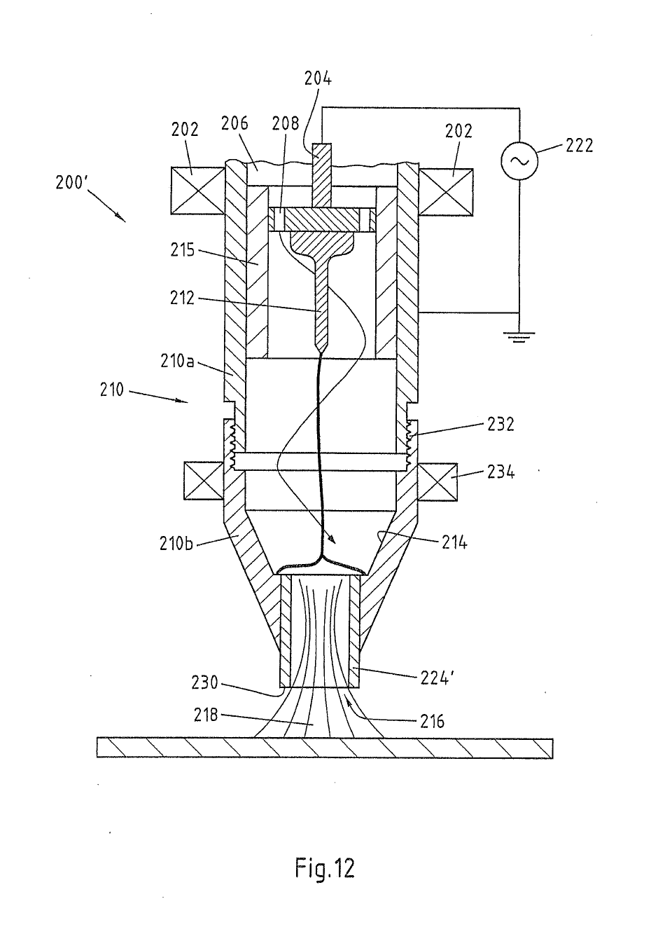

FIG. 12 a second embodiment of a plasma source with an axially displaceable outlet opening;

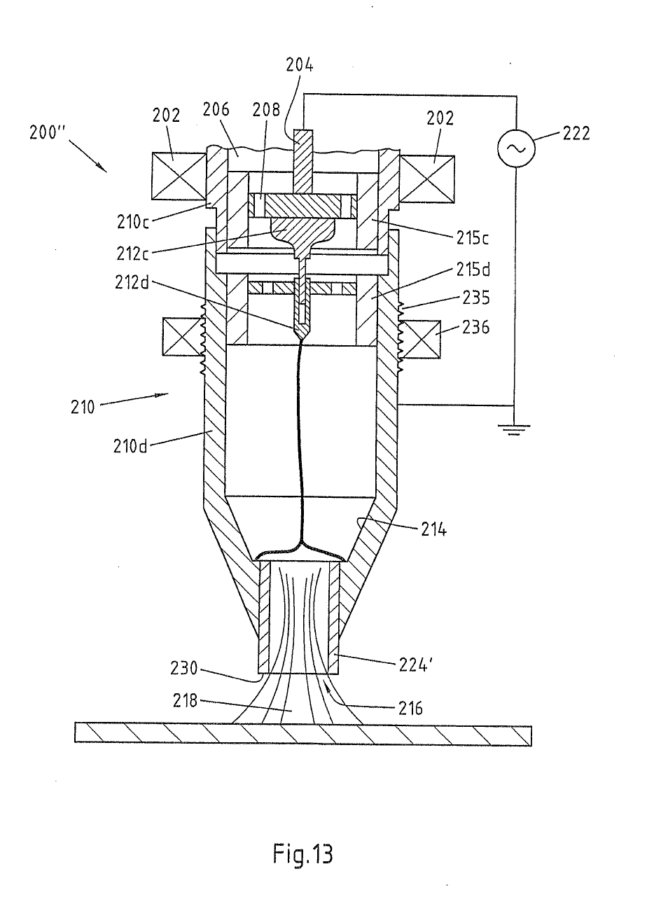

FIG. 13 a third embodiment of a plasma source with an axially adjustable outlet opening;

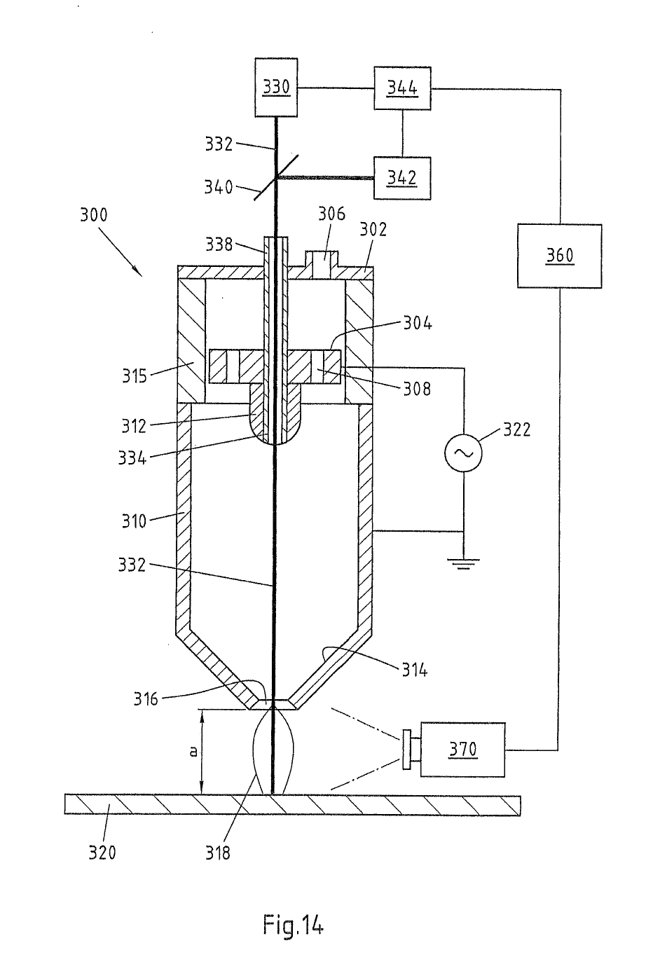

FIG. 14 a first embodiment of a plasma source with a distance checking device and

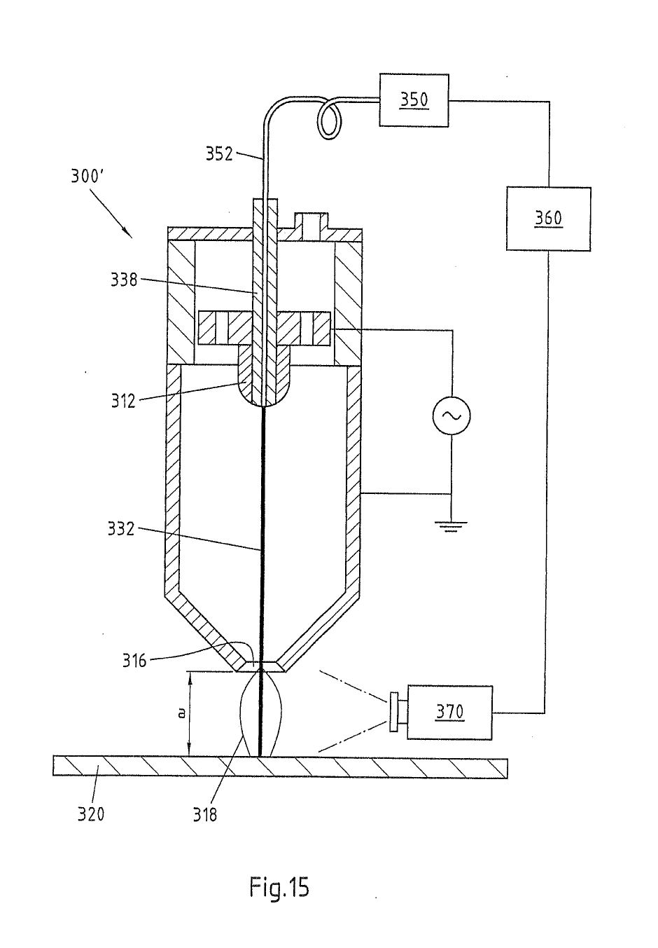

FIG. 15 a second embodiment of a plasma source with a distance checking device.

Before entering into a description of the device according to the invention for plasma treatment of living tissue embodiments of plasma sources will be described, that can be used with the device according to the invention. Here it is stressed that the plasma sources described are a certain type of plasma source. Similarly the invention is not limited to the use of these plasma sources.

A plasma source or plasma nozzle 10 shown in FIG. 1 has a housing or also a nozzle tube 12 in metal, which tapers conically to an outlet opening 14. At the end opposite the outlet opening 14 the housing 12 has a swirl device 16 with an inlet 18 for a working gas, for example air or nitrogen gas.

A dividing wall 20 of the swirl device 16 has a garland of bore holes 22 arranged transversally in the circumferential direction via which the working gas is swirled. The downstream, conically tapered part of the housing 12 therefore has the working gas flowing through it in the form of a vortex 24, the core of which follows the longitudinal axis of the housing 12. This vortex is shown schematically by a curved line 24.

On the underside of the dividing wall 20 an electrode 26 is centrally arranged, which protrudes coaxially into the housing 12. The electrode 26 is electrically connected with the dividing wall 20 and the other parts of the swirl device 16. The swirl device 16 is electrically insulated from the housing 12 by a ceramic pipe 30. By means of the swirl device 16 at the electrode 26 a high-frequency, high voltage, in particular alternating voltage or a high-frequency pulse DC voltage is applied, which is generated by a high-frequency transformer 32.

The primary voltage can be variably adjusted and is for example between 300 and 500 V. The secondary voltage can be between 1 and 5 kV or more, measured peak-to-peak. By way of example the frequency has an order of magnitude of between 1 and 100 kHz and is in particular also adjustable. The frequency can be set outside of the values indicated, provided that an arc discharge explained in the following occurs. The swirl device 16 is connected with the high-frequency generator 32 via a flexible high voltage cable 34. The inlet 18 is connected via a hose (not shown) with a pressurised working gas source with variable flow-rate, which in particular is combined with the high-frequency generator 32 to form a supply unit. The housing 32 is earthed.

Through the voltage applied a high-frequency discharge in the form of an electric arc 40 between the electrode 26 and the housing 12 is generated. “Electric arc” is the term used to describe the discharge phenomenon, since the discharge takes place in the form of an electric arc, although with direct current discharges the term electric arc is associated with essentially constant voltage values. Because of the in particular swirling flow of the working gas this electric arc is channeled in the core of the vortex along the axis of the housing 12, so that only when it reaches the outlet opening 14 does it branch off in the vicinity of the wall of the housing 12. The working gas which rotates in the area of the core of the vortex and thus in the immediate vicinity of the electric arc 40 at high flow speed, comes into intimate contact with the electric arc and is thereby to some extent converted into the plasma state, so that a jet 42 of atmospheric plasma, for instance in the shape of a candle flame, emerges from the outlet opening 14 of the plasma nozzle 10.

FIG. 1 shows the nozzle with a centred and essentially round outlet opening 14. Furthermore, it is also possible for the gas outlet to have a design that deviates from this. Thus FIG. 2 shows an outlet opening 14′ with an essentially slotted section, such that a flattened plasma jet 42′ is generated. The outlet opening 14′ here is formed by a separate mouthpiece 47′, which is connected with the housing 12′.

According to the embodiment after FIG. 3 the outlet opening 14″ is designed as a mouthpiece 47″ running transversally, and the mouthpiece 47″ or housing 12″ can be driven by a suitable drive in a rotating motion so that a transversal and rotating plasma jet 42″ is generated. In other words a rotating motion of the outlet 14 is achieved, whereby the plasma is swirled.

FIG. 4 further shows a plasma source in section for performing a plasma polymerisation. Here in the area of the outlet opening 14′″ a lancet 49 is provided, which downstream of the discharge 40′″ introduces a precursor material into the emerging plasma jet 42′″. The precursor material then reacts in the plasma jet and the depositing takes place of a defined layer on a surface which is simultaneously (pre)treated by the plasma jet 42′″.

There are various ways in which a precursor material can be introduced into a plasma source, so that the representation in FIG. 4 should only be taken as an example. The publications cited above contain further embodiments of this.

FIG. 5 shows a first embodiment of a device 50 for plasma treatment of living tissue with a plasma source 52 for generating an atmospheric plasma jet, which emerges in the shape of a curved flame at the front end of the plasma source. The device has a support device 54, which takes the form of a rest for a body 56 shown only schematically—comprising the tissue to be treated. The support device 54 can, however, have smaller dimensions if only one body part, such as an extremity, is to be plasma treated.

Furthermore, a movement device 58 for moving the plasma source 52 relative to the surface of the tissue, thus the body 56 is provided. The movement device works with three degrees of freedom and thus allows a 3-dimensional displacement of the plasma source 52. For each degree of freedom a linear drive 60, 62 and 64 is provided, wherein the individual linear drives allow the directions of movement identified by the double arrows x, y and z. Conventional drives are used as the linear drives.

The device 50 also has a control device 66 for controlling the movement device 58 and for controlling the operation of the plasma source 52. Thus the plasma source 52 can be adjusted, i.e. switched on and off especially, but can also be operated with differing plasma outputs. Similarly the movement device 58 is operated in such a way that the plasma source 52 is moved over the body 56, while the movement takes place in such a way that a predetermined distance range between the plasma source 52 and the surface of the body 56 is maintained.

The control device 66 shown has means for adjusting the plasma output as a function of the position relative to the tissue. For this purpose, for example, a keyboard and/or a pointer device (computer mouse) and a screen or a similar display device can be provided, on which at least sections of the area of the tissue to be treated are displayed. On the screen the user then determines which sections of the area are to be impinged upon by the plasma output. This plasma output profile is stored with the help of storage means and during the plasma treatment of the tissue the output profile is called up and adjusted as a function of the position of the plasma source relative to the tissue to be treated. For this at least one of the stated parameters is used.

FIG. 6 shows a second embodiment of a device 70 according to the invention for the plasma treatment of living tissue with a plasma source 72 for generating an atmospheric plasma jet 74.

On a flat table 76 a support device 78 is positioned for a body part 80 comprising the tissue to be treated, shown here schematically as a round arm in cross-section. The support device 78 is in this case adapted to the shape of the body and therefore results in a stabilising or partial fixing of the body part 80.

A movement device 82 for moving the plasma source 82 relative to the surface of the body 80, thus the tissue, has a curved guide 84, along which the support 86 for the plasma source 82 is arranged in a movable fashion. With the help of a drive (not shown) the support 86 can be moved along the curved guide 84 and perform a curved, preferably circular, movement. In this way the plasma source 82 can be passed around the body part 80 and adopt various angular positions. This movement is identified by the double arrow a. Furthermore a linear displacement (not shown in the detail) is possible with a drive that moves the plasma source 72 radially, and this is shown by the double arrow b. Furthermore the plasma nozzle 72 can be rotated in the support 86 in the plane of the curved guide, as identified by the double arrow c. In this way a position that deviates from a purely radial angle can be adopted by the plasma source 72.

This embodiment also has a control device 88 for controlling the movement device 86 and for controlling the operation of the plasma source 72. By means of corresponding lines 90 and 92 the control instructions are transmitted to the movement device 82 and the plasma source 72.

Furthermore, on the device shown in FIG. 6 a linear guide 94 is provided as part of the movement device 82, which allows a transversal movement of the curved guide 84 and thus a further degree of freedom. Thus the plasma treatment can take place not only essentially in the plane of the curved guide 84, but the movement of the plasma source 72 can also extend over a larger section of the body part, thus beyond the drawing plane of FIG. 6. For automatic displacement the linear guide 94 has a drive (not shown in the detail) which is connected via a line 96 with the control device 88.

FIG. 7 shows the embodiment presented in FIG. 6 in a perspective view, wherein the same reference signs identify the same elements as shown in FIG. 6. The linear drive 94 described above allows a movement transversally to the plane of the curved guide 84, with the direction of movement being shown in FIG. 7 by the double arrow d.

A further degree of freedom of movement of the plasma source 72 is shown by the double arrow e. By means of a suitable rotary drive as part of the movement device 82 the plasma source 72 can be positioned such that the direction of the plasma jet has a component in the direction d of the linear displacement. With sufficient radial displacement in direction b and a displacement in direction e therefore the end of a body part, for example the underside of a foot, the top of a head or other difficult to access areas of the body, can be treated with plasma.

FIG. 8 and FIG. 9 show further embodiments of a device according to the invention for plasma treatment of living tissue. The design of these embodiments corresponds essentially with the design that is shown in FIG. 6 and FIG. 7. Therefore the same reference signs identify the same element, as described above.

First of all, FIG. 8 shows how the support device 78 has a positioning device 100 for fixing the body part 80. The positioning device 80 is in this case in the form of a template, which encompasses the schematically shown arm 80 and thus fixes its position. The area of the tissue of the arm 80 to be treated is exposed by a window opening 102. For the treatment of various areas of a body part 80 then either a plurality of different templates is available, or the template 100 has a variable design and the position of the window opening 102 can have a variable and adjustable setting.

A further measure for improving the device consists of the positioning device having at least one switch 104—shown schematically in FIG. 8—which opens after a minimum movement of the body part 80. In place of the switch 104 a number of switches can also be provided on the template 100.

Similarly, in place of the switch 104 at least one movement sensor 106 shown in FIG. 9 can be provided which detects movement of the arm 80 in a contact-free, for example capacitive, inductive or optical manner. In particular if the template 100 is not provided, the contact-free movement sensor 106 can trigger the switching process described above. The movement sensor 106 can of course also be used when the template 100 according to FIG. 8 is applied.

In the event of excessive movement of the body part the switches 104 and 106 described above generate a switching signal and via a line 108 the control signal is transmitted to the control device 88. Since as a result of an excessive movement amplitude of the body part 80 an incorrect plasma treatment could arise, the control device 88 can use the switching signal from the at least one switch 104 or 106 in order to interrupt the plasma treatment and to operate the movement device 82 and/or the plasma source 72 in such a way that no damage to the tissue can arise.

As shown in FIG. 8 and FIG. 9 a housing 110 is provided in which the plasma source 72 and the movement device 82 are arranged. In this way the plasma treatment is carried out in an at least in part screened area spanning the housing 110. The representation in FIG. 8 and FIG. 9 shows a tunnel-shaped housing 110, which is open on two sides. The housing can, however, also essentially be closed on all sides and either allow only a passage of the body part 80 to be treated or accommodate the entire body of a patient.

In addition the housing 110 can have a suction device (not shown), in order to draw off the process gases used in or resulting from the plasma treatment.

FIG. 9 also shows how a temperature control device 120 checks the temperature of the tissue to be treated. For this purpose the temperature control device 120 has a temperature sensor 122, which either measures the temperature in the area of the body part being plasma treated, or though the detection of the thermal radiation on the body surface measures the exact temperature of the tissue being treated. The temperature control device 120 is connected via a line 124 with the control device 88.

FIG. 10 shows a further alternative for the design of a device according to the invention for plasma treatment of living tissue, in which a movement device 130 with a robot arm 132 is used. The robot arm 132 allows six degrees of freedom in the movement of the plasma nozzle 134 relative to a body to be treated, which is lying on a bed 136. The movement device 130 with its free access can therefore in particular be used in operations in order to treat the tissue to be operated on before, during or after the operation with plasma. Even if the robot arm 132 is shown without a surrounding housing, it is basically also possible to arrange a freely movable robot arm 132 within a housing. For the control of the device a control device 138 is provided for.

The devices shown in FIGS. 5-10 for plasma treatment of living tissue each have a plasma source 52, 72 or 134. The devices are not limited to the application of just one plasma source 72; thus two or more plasma sources 72 can also be provided for which have different designs and fields of application. So in particular the plasma sources shown in FIGS. 1-4 are suitable for one use. Thus in particular the jet shaping (FIG. 1 and FIG. 2), the expansion of the surface to be treated (FIG. 3) or the plasma polymerisation (FIG. 4) can be applied.

Additionally and in particular the configurations of the plasma sources shown below in FIGS. 11-15 can be used in an advantageous manner in the device according to the invention for plasma treatment of living tissue. Nevertheless, the area of use is not limited to the plasma treatment of living tissue. Therefore in the following description the term “object” is used in place of “body”, “body part” or “tissue”.

FIGS. 11-13 show embodiments of plasma nozzles with a variably adjustable length for setting the distance between the plasma source and the object. Here a distinction has to be made between the movement devices according to FIGS. 5-10, which bring about a movement of the entire plasma source, that is to say a support for the plasma source in the longitudinal direction, and a device that only varies the length of the plasma nozzle.

FIG. 11 shows a plasma source 200 for generating an atmospheric plasma jet with a design similar to the embodiments of FIGS. 1-4.

The plasma source 200 has a support 202 which is connected with the housing 210 and can be moved with the plasma source 200 as a whole. Via the support 202 the feed for the electrical supply by means of an electrical connection 204 and gas feed 206 via a gas inlet 208 can be set up.

Furthermore the plasma source 200 has a housing 210, an internal electrode 212 and an external electrode 214 that is at least in part formed in the housing 210, which by means of the insulation 215 is electrically insulated from the internal electrode 212. On the underside of the housing 210 an outlet opening 216 is formed from which the plasma jet 218 emerges and impinges on the object 220. The plasma jet 218 is shown here more in the form of radiation lines and not in the form of a round flame. The internal electrode 212 and the outlet opening 216 together define an axial direction.

The plasma source 200 also has means for applying a high-frequency, high voltage between the internal electrode 212 and the external electrode 214 in the form of a voltage source 222 and corresponding supply lines. The way in which the plasma source operates here is essentially identical to the explanation provided above using FIG. 1.

According to the invention, at the bottom end of the housing 210 a mouthpiece 224 and a rotary drive 226 which is connected with the housing 210 are provided. The mouthpiece 224 has a thread 228 on the outside which engages with the rotary drive 226. Similarly a spindle drive, through the operation of the rotary drive 226, can vary the position of the external end 230 of the outlet opening 216 or the mouthpiece 224 in the axial direction relative to the support 202. In other words, the housing 210 is length-adjustable in the area of the outlet opening 216.

If the thread is selected with a large pitch, then the mouthpiece can be moved back and forth with a smaller rotation of the rotary drive 226 rapidly in the axial direction. Here the movement of the mouthpiece 224 can be performed more quickly because of the lower mass than if the entire housing 210 or the entire plasma source 200 were to be moved.

FIG. 12 shows a further configuration of the plasma source described above wherein the same reference signs identify the same elements as in FIG. 11.

Unlike the representation in FIG. 11 the housing 210 is variable in length between the internal electrode 212 and the outlet opening 214. To that end the housing 210 is in two parts and has an upper housing part 210a and a lower housing part 210b, which are connected together by means of a thread 232 in the overlapping area. A rotary drive 234, which is secured against rotation in relation to the support 202 engages with the lower housing part 210b. By operating the rotary drive 234 the lower housing part 210b is rotated in relation to the upper housing part 210a and displaced in the axial direction by means of the thread 232.

This allows the position of the external end 230 of the outlet opening 224′ to be varied in the axial direction relative to the support 202. The advantage of this configuration is that the internal area of the housing 210 provided for the discharge in particular at the lower end in the area of the mouthpiece 224 is not altered, so that the discharge conditions change less than with the configuration according to FIG. 11. The mass to be moved is indeed greater, but is still considerably less than if the entire plasma source 200′ were to be moved.

FIG. 13 shows a further embodiment of the plasma source described above wherein the same reference signs identify the same elements as in FIG. 11 and FIG. 12.

Unlike the representation in FIG. 11 and FIG. 12 the plasma source is designed so that the position of a housing part 210d together with an internal electrode part 212d can be varied relative to the support. To that end the housing 210 has an upper housing part 210c and the lower housing part 210d. Similarly the internal electrode 212 comes in two parts and has an upper electrode part 212c and the lower electrode part 212d. The insulation 215 is similarly divided up into an upper insulation part 215c and a lower insulation part 215d.

The lower housing part 210d is connected via the lower insulation part 210d with the lower internal electrode part 212d. The two internal electrode parts 215c and 215d are connected together in a telescopic fashion, wherein the electrical conductivity must be maintained. The two housing parts 210c and 210d are similarly inserted into each other by means of a telescopic arrangement. So the unit comprising the lower parts 210d, 215d and 212d can be displaced relative to the upper parts 210c, 215c and 212c of the plasma source 200″.

The outside of the lower housing part 210d is provided with an external thread 235, which engages with the rotary drive 236. Similarly, through the operation of the rotary drive 236 the position of the lower housing part 210d can be varied relative to the upper housing part 210c or the support 202, so that the end 230 of the outlet opening 216, thus the mouthpiece 224 is displaced in the axial direction relative to the support 202.

Even if with the present embodiment the mass to be moved is greater than with the two embodiments described previously, the reduction in weight is still sufficient to allow a high speed of displacement to be achieved. The advantage of this configuration is in any case that the entire geometry of the discharge area between the front end of the internal electrode 212 and the front end of the housing 210 or of the mouthpiece 224′ does not vary, even though the length of the plasma source is changed.

In the following using FIG. 14 and FIG. 15 two embodiments with an integrated distance control device are described.

FIG. 14 shows a plasma source 300 for generating an atmospheric plasma jet with a comparable design to the embodiments according to FIGS. 1-4 and 11-13. To aid clarity in FIG. 14, unlike in the other figures, the air flow or the vortex and the discharge channel or the arc discharge are not shown.

The plasma source 300 has a support 302 with which the entire plasma source 300 can be moved, for example by means of a movement device in a device according to one of FIGS. 5-10. By means of the support 302 the feeding of the electrical supply via an electrical connection 304 and the gas feed 306 via a gas inlet 308 can also be set up.

Furthermore the plasma source 300 has a housing 310, an internal electrode 312 and an external electrode 314 that is at least in part formed in the housing 310, which by means of the insulation 315 is electrically insulated from the internal electrode 312 and which like all the other embodiments is earthed. In this case the insulation 315 constitutes an extension of the housing 310. On the underside of the housing 310 an outlet opening 316 is formed from which the plasma jet 318 emerges and impinges on the object 320. Here the plasma jet 318 is shown in the form of a round flame, similar to a candle flame. The internal electrode 312 and the outlet opening 316 together define an axial direction.

The plasma source 300 also has means for applying a high-frequency, high voltage between the internal electrode 312 and the external electrode 314 in the form of a voltage source 322 and corresponding supply lines. The way in which the plasma source 300 operates here is essentially identical to the explanation provided above using FIG. 1.

According to the invention the plasma source 300 shown in FIG. 14 means for coupling a laser beam in the axial direction are provided and optical means for measuring the distance between the front end of the outlet opening 316 and the object 320 to be treated, wherein a signal from the reflection of the laser beam from the surface of the object 320 is evaluated.

A laser source 330 generates a laser beam 332, which is directed so that it runs through a channel 334 formed in the internal electrode 312. For this purpose in particular an insulating tube 338 is provided which extends to the outside of the support 302. The laser source 330 is adjusted so that the laser beam 332 in particular runs essentially along the axis, through the plasma source 300 and through the outlet opening 316 out of the plasma source 300. The adjustment of the laser source 330 and the configuration of the inter electrode therefore constitute in the present embodiment the means for coupling the laser beam.

The laser beam impinges upon the surface of the object 320 and is to some extent reflected back in the opposite direction along the previously described light path.

By means of an output coupling mirror 340 the reflected component of the laser beam 332 is reflected onto a photosensor 342, so that a measured signal is recorded and transmitted to a control and evaluation unit 344. In a prior art manner the control and evaluation unit 344 intensity modulates the laser beam and the modulation of the laser light is determined in the context of a propagation time or phase length measurement between the radiated and measured laser light. Thus a laser distance measurement of a prior art is integrated with an electronic distance measurement in a plasma source.

From the measured distance the distance a of interest between the front end of the outlet opening 316 and the surface of the object 320 is inferred. For the other light run lengths are known and can be deducted from the measured distance. Similarly the change in distance a can be determined by a differential evaluation of the measured signals.

The output signal from the control and evaluation unit 344 is then transmitted as a distance control signal to a control device 360 for further processing. For example, the distance control signal can be used for controlling the distance between the plasma source and an object, in order in the event of a movement of the plasma source along the surface of the object to maintain a specified distance a within a specified tolerance.

FIG. 15 shows a plasma source 300′, with a design essentially corresponding with the embodiment shown in FIG. 14. Here the same reference symbols identify the same elements.

A distance control device 350 is provided for first of all which features a laser. The laser beam is then injected into fibre optics 352.

The fibre optics 352 then constitute the means for coupling the laser beam 332, wherein the fibre optics 352 run through the internal electrode. Here the fibre optics are preferably accommodated in the insulating pipe 338. In FIG. 15 the fibre optics 352 run as far as the front end of the pipe 338 and the internal electrode 312, but the fibre optics 352 can also be set back slightly.

At the outlet of the fibre optics 352 the laser beam emerges and impinges on the surface of the object 320. The fibre optics 352 also recapture the partially reflected laser light and transfers this back to the distance control device 350, where the reflected light is decoupled and applied to an optical sensor. Here in the same way the distance control signal is then generated, as described by reference to FIG. 14. The distance control signal is then transmitted via a line to the control device 360 for further processing.

In FIG. 14 and FIG. 15 a camera 370 is also shown, wherein by means of imaging the distance a between the front end of the outlet opening 316 and the surface of the object 320 is monitored. The camera 370 also comprises an evaluation unit for generating a distance control signal, which is transmitted via a line to the control device 360. The camera can be used instead of or in addition to the laser distance measurement.

The various embodiments described above of the plasma sources according to FIGS. 11-15 can be used in the device according to the invention for plasma treatment of live tissue. Thus the distance control device described can check the distance between the plasma source and the tissue and the length of the plasma source can be set in order to regulate the distance between the front end of the plasma source and the tissue.

In general terms, then, the control device controls the operating parameters of the plasma source and the movement device as a function of at least one of the preset parameters of plasma output, distance, temperature, tissue type and desired effect.

1. A device for plasma treatment of living tissue, comprising:
a plasma source for generating an atmospheric plasma jet,
a support device for a body part comprising the tissue to be treated,
a movement device for moving the plasma source relative to the surface of the tissue, and
a control device for controlling the movement device and for controlling the operation of the plasma source,
wherein the control device has means for adjusting the plasma output as a function of the position relative to the tissue.

2. The device according to claim 1, wherein the support device has a positioning device for fixing the body part.

3. The device according to claim 2, wherein the positioning device has at least one switch or movement sensor, which after a minimum movement of the body part generates a switching signal.

4. The device according to claim 1, wherein a distance control device checks the distance between the plasma source and the tissue.

5. The device according to claim 1, wherein the plasma source has a variably adjustable length for setting the distance between the plasma source and the tissue.

6. The device according to claim 1, wherein a temperature control device checks the temperature of the tissue to be treated.

7. The device according to claim 1, wherein particular tunnel-shaped housing is provided in which the plasma source and the movement device are arranged.

8. The device according to claim 1, wherein the control device has means for establishing an output profile within the area to be treated.

9. The device according to claim 1, wherein the control device has means for determining the absolute position of the tissue.

10. The device according to claim 1, wherein the control device has means for determining the position of the tissue relative to the plasma source during the movement and operation of the plasma source.

11. The device according to claim 1, wherein means for performing plasma polymerisation to generate a wound seal and/or of a medication are provided for, wherein with the help of a plasma, a precursor material is reacted and the reacted product deposited onto the surface.

12. The device according to claim 1, wherein the plasma source for generating an atmospheric plasma jet has a support, a housing, an internal electrode, an external electrode that is at least in part formed in the housing, a gas inlet, an outlet opening formed on the housing and means for application of a high-frequency high voltage between the internal electrode and the external electrode, wherein the internal electrode and the outlet opening together define an axial direction, wherein the position of the external end of the outlet opening can be varied in the axial direction relative to the support.

13. The device according to claim 12, wherein the housing in the area of the outlet opening is length-adjustable or in that the housing is length-adjustable in the axial area between the internal electrode and the outlet opening or in that the position of a housing part together with an internal electrode part is variable relative to the support.

14. The device according to claim 1, wherein the plasma source for generating an atmospheric plasma jet has a housing, an internal electrode, an external electrode that is at least in part formed in the housing, a gas inlet, an outlet opening formed on the housing, means for application of a high-frequency high voltage between the internal electrode and the external electrode,
wherein the internal electrode and the outlet opening together define an axial direction,
wherein means for coupling a laser beam in the axial direction are provided, and
wherein means optically measure the distance from the front end of the outlet opening to the object to be treated, wherein a signal from the reflection of the laser beam from the surface of the object is evaluated.

15. The device according to claim 14, wherein the means for coupling the laser beam take the form of a channel created in the internal electrode or in that the means for coupling the laser beam take the form of fibre optics passing through the internal electrode.
